(19) United States
(12) Patent Application Publication
(10) Pub. No.: US 2006/0184098 A1
(43) Pub. Date: Aug. 17, 2006

(54) Subarachnoid spinal catheter for transporting cerebrospinal fluid

(75) Inventors: James Barnitz, Schwenksville, PA (US); David Hesson, Malvern, PA (US); Glenn = Frazer, Wynnewood, PA (US); Timothy Pelura, Malvern, PA (US)

Correspondence Address: LAW OFFICES OF ARTHUR E. JACKSON, P.O. BOX 88, HOPEWELL, NJ, 08525, US

(73) Assignees: Neuron Therapeutic, Inc., Malvern, PA (US); Integra LifeSciences Corporation, Plainsboro, NJ (US)

(21) Appl. No.: 10/543,732

(22) PCT Filed: Apr. 21, 2003
(86) PCT No.: PCT/US03/12171

§ 371 Date: Sep. 12, 2005

Related U.S. Application Data
(60) Provisional application No. 60/373,954, filed on Apr. 19, 2002.

(51) Int. Cl. A61M 3/00

(52) U.S. Cl. 604/43

(57) ABSTRACT

Provided is a multi-lumen intrathecal catheter capable of use for circulating a fluid through a portion of the spinal cord of a patient and having an insertable portion adapted for insertion into the spinal cord, comprising: a first lumen having a distal and a proximal end, the proximal end being adapted to being connected to a reservoir containing a fluid; a first fluid exchange section connected to the distal end of the first lumen having at least one inlet; a second lumen having a distal and a proximal end; and a second fluid exchange section connected to the distal and of the second lumen having at least one outlet, wherein at least one inlet and at least one outlet are offset from each other.

This invention is in the field of neurosurgery and in particular, it is directed to a catheter for transporting fluid to and from the subarachnoid space of the spine and brain and to a method of treating neural tissue injury by delivering one or more of therapeutic drugs, nutrients, and oxygen, cooling or heating fluid in the subarachnoid space, or removing one or more of blood, blood products, cellular metabolites, or edematous fluids.

The brain and spinal cord are neural tissues of the central nervous system and are surrounded by cerebrospinal fluid (CSF). The CSF, which has a composition similar to that of plasma, cushions the brain and spinal cord against external traumatic forces applied to the head, neck, spine or body. The CSF, therefore, lessens primary injuries resulting from spine, neck, and head trauma. The etiology of neural tissue injury includes primary injuries induced by accidental trauma, surgical trauma, or both. Neural tissue injury may also result from blood surrounding the spinal cord or brain, resulting from trauma or hemorrhage, which may cause vasospasm that results in neural tissue ischemia and damage.

Spinal and brain tissue injuries are significant injuries because they can lead to neural tissue ischemia and ultimately, neurological deficit such as paralysis or death. Since the spinal cord and brain are enclosed within the bony spine and skull and are covered by a relatively inelastic membranes, there is relatively little room for the spinal cord or brain to expand. Due to this closed space environment, swelling of spinal cord or brain tissue secondary to injury compresses neural tissue blood vessels. The neural tissue then becomes ischemic (secondary injury), initiating a cascade of events, leading to yet more tissue edema and swelling. Severe cases of neural tissue injury are life-threatening or result in motor or sensory dysfunction.

Currently, there is no available treatment that effectively minimizes neural tissue injury or vasospasm after trauma. Surgical procedures such as spinal decompression and stabilization treat primary injuries only, and are often detrimental, delaying overall patient recovery due to the overwhelming physical stress of the operations. Long operative times and exposure to general anesthesia also increase the risk of intraoperative and postoperative complications.

Various drugs have been investigated for the treatment of spinal cord injury. For example, methylprednisolone, a corticosteroid, is standardly infused as a continuous intravenous drip after spinal cord injury, and has been advocated to improve functional (motor) recovery. However, clinical improvements are minimal when compared to control subjects. Side effects such as GI bleeding and increased rates of wound infection have also been demonstrated in some studies.

Various synthetic artificial cerebrospinal fluids (ACSF) have also been developed to treat nervous system diseases. For example, an ACSF with an oxygen carrying component, e.g. a fluorocarbon (oxygenated fluorocarbon nutrient emulsion (OFNE)), is described in U.S. Pat. Nos. 4,393,863, 4,446,155, 4,686,085, 4,378,797, and 4,981,691 to treat ischemic tissue in stroke patients. Drugs useful for treating injury, such as antiinflammatory steroids, may be added to the ACSF. Thrombolytic drugs useful for breaking up blood clots, such as tissue plasminogen activator, streptokinase, and other agents may also be added to the ACSF as part of the treatment.

Various devices have also been developed to treat neural disease. For example, U.S. Pat. No. 4,904,237 to Janese discloses an apparatus for the exchange of ACSF; U.S. Pat. Nos. 4,085,631; 4,994,036; 5,234,406 describe kits for administering spinal subarachnoid anesthesia; U.S. Pat. No. 5,738,650 to Greg discloses a subarachnoid needle to administer agents to the subarachnoid space; and U.S. Pat. No. 4,767,400 to Miller et al. is directed to a porous ventricular catheter that drains ACSF.

Catheters have been also been described for the exchange of fluids. A dual-lumen suction catheter for removing fluids from the stomach of a patient has been described by Linder, U.S. Pat. No. 5,931,831, but would not be suitable for introduction into spinal spaces. Prosl, U.S. Pat. No. 5,868,717, describes a dual lumen vascular catheter designed for high volume, >250 mL/min, flow of fluids related to hemodialysis. This catheter is too large and flow too great to be useful in the application of fluids to the spinal space. Love, U.S. Pat. No. 6,221,622 B1, describes a dual-lumen catheter used for purposes of obtaining cellular materials from human breast ducts. The spacing of the outlets of the two lumens at the distal end of the catheter is too close (4.06 mm) to be useful for treatment of spinal cord injuries.

Thus, as described above, various techniques including the injection of ACSF fluid into the CSF pathway have been proposed for treating neural tissue injury. Notwithstanding these prior techniques, new apparatuses having the features of the present invention are needed for treating neural tissue injury.

### SUMMARY OF THE INVENTION

The present invention includes multilumen catheters and kits of multilumen catheters for fluidly communicating with the subarachnoid space that are useful for the treatment of neural tissue damage of the spine, neck, and basal cisterns of the brain. Fluid, such as, for instance, an ACSF is transported to and from the cerebrospinal fluid pathway using a multilumen catheter having a size and flexibility suitable for entry into the subarachnoid space in the spine without inducing substantial trauma to the neural tissue. The catheter can in certain embodiments be used to administer therapeutic agents or remove deleterious agents along with the ACSF. The catheter is preferably maneuverable within the subarachnoid space. The catheter can also in some embodiments contain sensing devices to monitor physiologic parameters such as pressure and temperature or have fiber optic viewing capability. An advantage of the multilumen catheter is that only one entry point is required in many embodiments. Minimizing the point of entry reduces the likelihood of infections and reduces the associated risks of inserting additional spinal needles.

One embodiment of the present invention includes a multilumen catheter for transporting fluid such as an ACSF. In this embodiment, the multilumen catheter features an elongated tubular member having one lumen for delivering fluid and a second lumen for withdrawing fluid from the subarachnoid space. The lumens are separated by a shared partition and each lumen terminates in a fluid exchange section with an at least one opening. The opening or set of openings of each fluid exchange section is separated in this embodiment by a minimum of 30 mm (measured from the closest openings) from the opening or set of openings of each other fluid exchange section in order to allow the ACSF to flow over a site of spinal injury.

These and other variations and features of the invention will become more apparent when the following detailed description of the invention is read in conjunction with the accompanying drawings.

### BRIEF DESCRIPTION OF THE DRAWINGS

FIG. 1 is a cross-sectional view of the central nervous system head and spine.

FIG. 2 is a cross-sectional view of the spine, site of injury, and a catheter.

FIG. 3 is a cross-sectional view of an intrathecal dual lumen catheter in accordance with the present invention.

FIG. 4 is a cross-sectional view of an intrathecal catheter in accordance with the present invention.

FIGS. 5a-b are cross-sectional views of an open tip catheter with guide wire and a catheter with additional lumen for a sensor.

FIG. 6a-d is a cross-sectional view of fenestrations on intrathecal catheters.

FIG. 7 is an illustration of an intrathecal catheter kit accessing the subarachnoid space from different regions along the cerebrospinal fluid pathway.

### DETAILED DESCRIPTION OF THE INVENTION

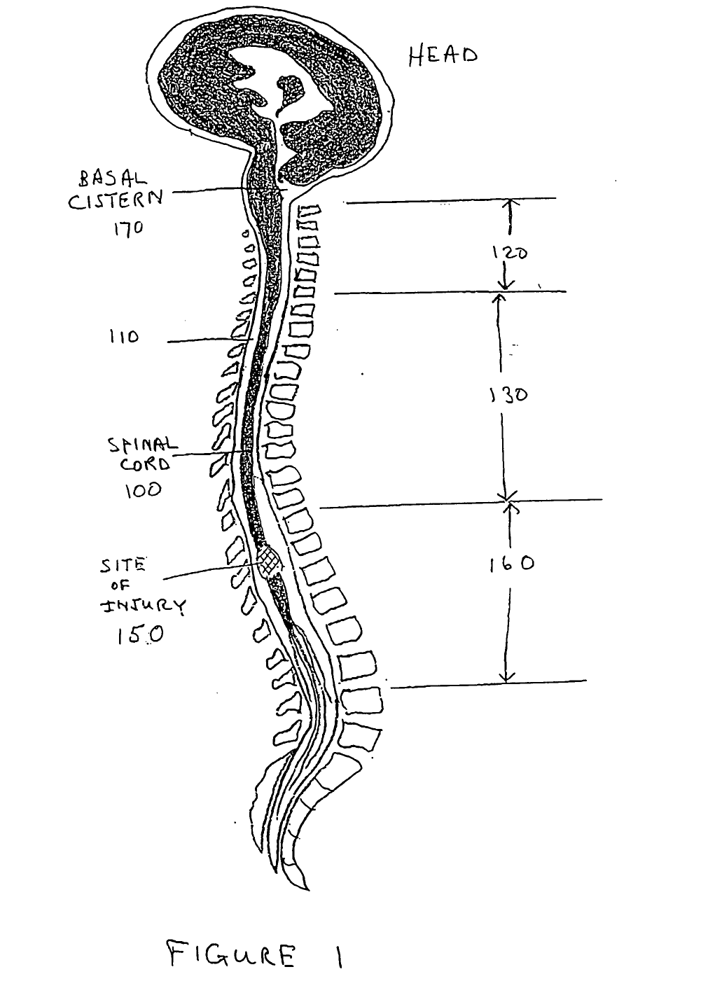

Prior to describing specific features of the present invention, a brief review of the pertinent anatomy is provided. Referring to FIG. 1, the inter-connection between the brain, spinal cord 100, and the subarachnoid CSF pathway is shown. Subarachnoid space 110 surrounds the brain and spinal cord 100 and comprises various regions which include the cerebral subarachnoid space 120, and cervical spinal subarachnoid space 130, the thoracic spinal subarachnoid space 140, and the lumbar spinal subarachnoid space 160. A potential site of injury 150 to the spinal cord is shown for reference. This site of injury could occur anywhere along the length of the spinal cord. Basal cistern 170 is shown as a site where blood from a damaged brain or spinal cord may collect resulting in possible cerebral vasospasm and further neural tissue injury.

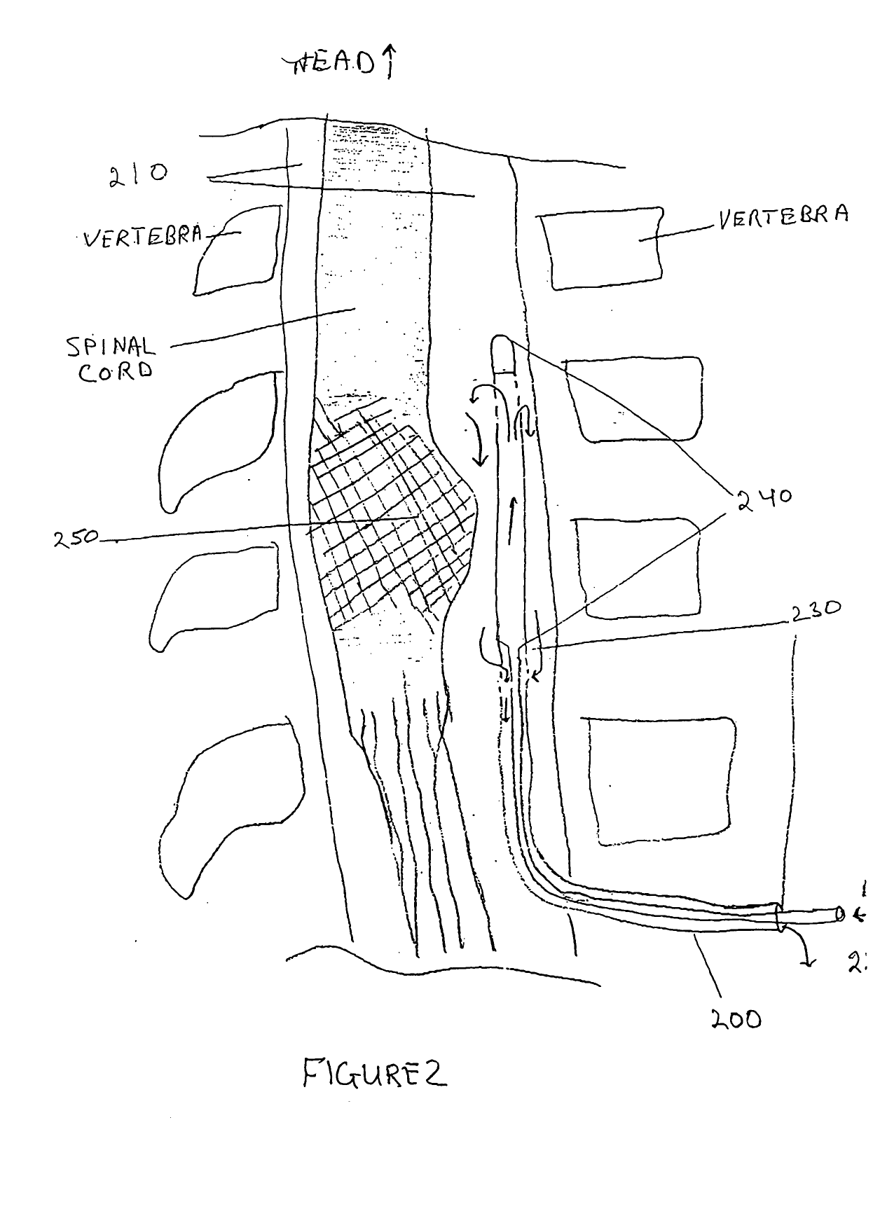

As illustrated in FIG. 2, the present invention includes a kit containing a dual-lumen catheter 200 for transporting fluid, such as an artificial cerebrospinal fluid, to above the site of injury and from below the site of injury in the subarachnoid space 210. The pressure at the entrance to the inflow pathway 210 is limited in certain embodiments to 22.5 psi to be compatible with standard barbed fittings. The pressure loss across the outflow pathway 130 is preferably limited to less than 35 mmHg to be compatible with physiologic pressure. The catheter 200 has a size and a flexibility that allow suitable entry into the subarachnoid space without substantial trauma to the neural tissue. Safely accessing the subarachnoid space requires navigating a number of tissues and provides only limited space for maneuvering a fluid delivery device such as an intrathecal or subarachnoid catheter. The maximum size of the extended inflow section 240 is limited to 14 gauge (2.03 mm) in the preferred embodiments. The minimum inside diameter of the base section inflow pathway is 0.38 mm in the preferred embodiments to allow use of a guide wire during catheter placement. Once positioned, fluid is delivered to the injured neural tissue site 250.

Preferably, cerebrospinal fluid or artificial cerebrospinal fluid is transported to the subarachnoid space. As used herein, the term artificial cerebrospinal fluid or ACSF includes organic cerebrospinal fluid, artificial cerebrospinal fluid, or a mixture of the two fluids. Most preferably, a hyperoncotic cerebrospinal fluid as disclosed in U.S. application Ser. No. 09/440,038 (filed Sep. 12, 1999), the entirety of which is incorporated herein by reference, is used in accordance with the present invention. However, the invention also includes the use of ACSF to treat conditions such as ischemia as described in U.S. Pat. No. 4,393,863 to Osterholm, the entirety of which is also incorporated herein by reference.

### First Embodiment of the Present Invention

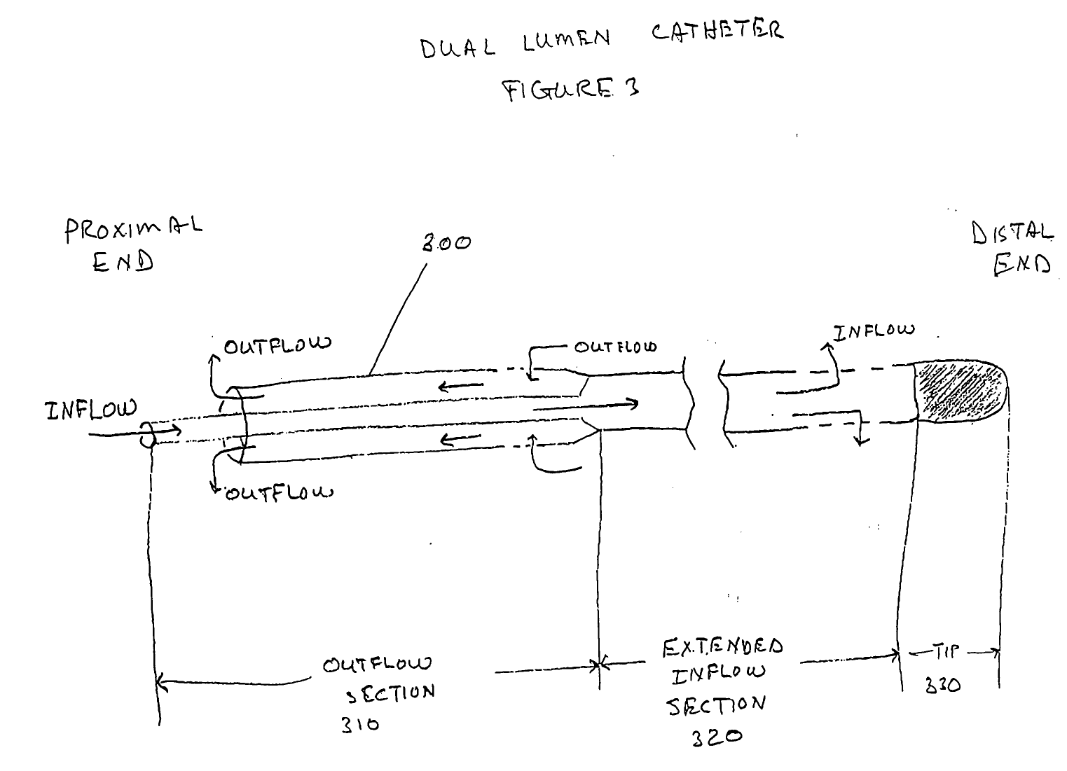

The present invention includes a kit for safely accessing and fluidly communicating with the subarachnoid space. The kit includes a catheter suitable for entering the lumbar space of the spine and advancing up the subarachnoid space in the spine. The catheter 300, as shown in FIG. 3, is an elongate member having a proximal end and a distal end.

The elongate tubular member has a size and flexibility suitable for entry into the subarachnoid space. That is to say, the elongate tubular member has a size and flexibility allowing it to be manipulated in the subarachnoid space without causing significant trauma to the neural tissue. The elongated tubular member 300 is comprised of an outflow section 310, an extended inflow section 320 (for flow into the CSF pathway), and a tip 330. A soft polymeric tip plug 330 helps to minimize trauma to neural tissue upon catheter insertion.

The dimensions of catheter 300 in an exemplary embodiment are as follows: overall length approximately 180-600 mm; outflow section 310 length approximately 150-300 mm; extended inflow section 320 length approximately 30-450 mm; and tip plug 330 length approximately 0.025-5.0 mm. The specific dimensions are selected to promote flow of ACSF into and out of the subarachnoid space while maintaining and not exceeding a safe physiologic continuous pressure of 35 mmHg within the subarachnoid space. Pressures greater than 35 mmHg for several seconds are measured in persons when they cough or strain, but are not frequent and are not sustained fro more than several seconds. The outflow section is of a length that allows safe entry into the lumbar region of the spine while preferably being as short as possible to minimize the resistance to flow within the outflow section. The overall diameter of the outflow section is preferably limited, such as to 12 gauge (2.64 mm) to be safer for entry into the lumbar space. The overall length of the extended inflow section can vary, such as from 30-450 mm depending on where the injury in the spine occurs. Specific examples of useful lengths of the extended inflow section include 30 mm or more, 60 mm or more, 90 mm or more, and 120 mm or more. The further distance up the spine from the lumbar region the longer the extended inflow section will typically be. The extended inflow section preferably has a larger cross sectional area than the inflow section contained in the outflow path in order to reduce resistance in inflow, thereby maintaining therapeutic flow at physiologic pressures. The extended inflow section has a diameter selected to avoid damage to tissue within the spine, such as of 12 gauge (2.03 mm) or less.

### Lumen Dimensions

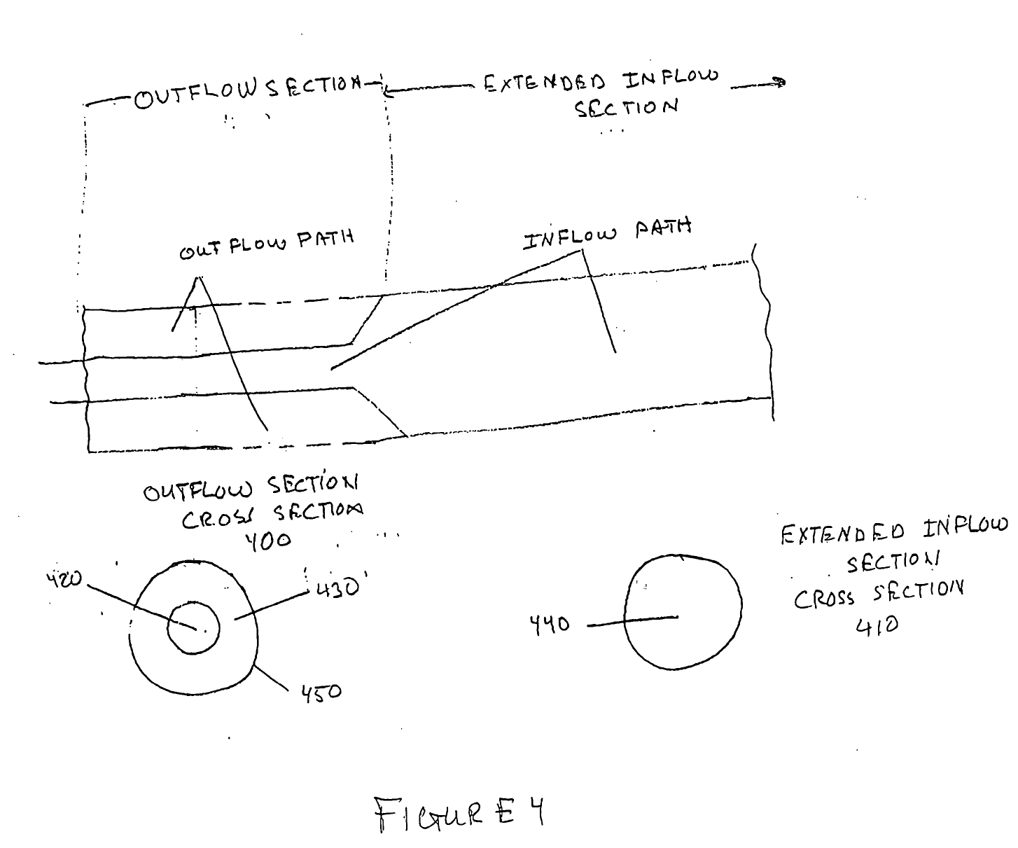

FIG. 4 illustrates cross sections of the outflow section 400 and extended inflow section 410. The size of inflow path 420 and outflow path 430 of outflow section 400 and the lumen of extended inflow path 440, the overall length of the catheter, and the size, placement, and number of fenestrations through the elongate tubular member wall are chosen so that during normal use, the catheter can accommodate the desired ACSF and included fluid or drug flow rates at physiologic subarachnoid pressures. Other features having an effect on the flow and pressure relationship are similarly selected. These dimensions are chosen and interrelated depending on the performance characteristics required.

Further, outflow section 400 can have a braid or coil within outer cover 450, or similar reinforcing feature. A braid or coil provides radial strength and elastic stability to ensure that the catheter resists kinking or crushing when passed through fibrous tissue such as the dura mater and spinal ligaments. A braid or coil (or other reinforcing feature) also resists kinking when being manipulated in the subarachnoid space. A braid can be woven or unwoven and braid and coil pitch can vary to control the stiffness of the outflow section of the catheter. A coil or woven braid can be made, for example, from one or a combination of several metals, such as platinum, palladium, rhodium, gold, tungsten, titanium, tantalum, and nickel, alloys thereof, stainless steel, and polymers. Braid and coil components, materials of construction, and method of production can be designed similar to the lumbar drainage catheter described in U.S. application Ser. No. 09/382,136, (filed Nov. 26, 1999) the entirety of which is herein incorporated by reference.

The optimal dimensions of the catheter are preferably based upon attaining maximum flow rate while not compromising the structural integrity of catheter assembly 300 or exceeding safe physiologic pressures. For applications in the CSF pathway, in the preferred embodiments, the catheter should accommodate flow rates of between about 1-80 mL/min, preferably between about 5 and 60 mL/min, more preferably 10-40 mL/min, and most preferably about 20 mL/min.

Any biocompatible, polymeric material suitable for medical application that meets the requirements of the catheter assembly can be used. Because this catheter can be used with the above noted fluids, the polymers chosen should be compatible with those fluids, some of which contain a significant amount of fluorocarbon. Particularly useful classes of polymers are polyurethanes, various polyethylenes (including low density polyethylene (LDPE), linear low density polyethylene (LLDPE)), polypropylene, polybutenes, polyamide (such as Nylons), high density polythethylene (HDPE), polyimides, polyvinylchloride, fluorocarbon resins (e.g., PTFE, FEP, vinylidene fluoride, their mixtures, copolymers, block copolymers, etc.), and other polymers of suitable hardness and modulus of elasticity. Blends, alloys, mixtures, copolymers, and block copolymers of these materials are also suitable if desired. Medical grade urethane sold under the name TECOFLEX (Thermedics, Inc., Waltham, Mass.) having a Shore hardness of approximately 80 A-200 A, preferably about 95 A-125 A, is suitable.

A lubricious coating on the exterior surface of the tubular member wall is also useful to help minimize tissue trauma upon catheter insertion. The portions or the catheter that come in contact with tissue can be coated with a lubricious (and typically hydrophilic) layer, which either is chemically bonded or coated onto the catheter exterior surface. Descriptions of suitable procedures for producing such lubricious coatings are found in U.S. Pat. No. 5,531,715 to Engelson et al. and U.S. Pat. No. 5,538,512 to Zenzen et al., the entirety of each of which is incorporated by reference. A preferred hydrophilic coating is a polypyrrolidone-based material sold by Hydromer Co. Although less preferred, silicone oils such as MDX are also suitable.

### Extended Inflow Section

Extended inflow section 320 is preferably made from a polymeric material as described above with respect to the proximal section 310. A notable preferred difference between extended inflow section 320 and outflow section 310 is the lower elastic modulus and/or hardness of distal section 320, which is desired so that the extended inflow section 320 can be adequately maneuvered into the subarachnoid space without significant trauma to neural tissue. Applicants have found that TECOFLEX medical grade urethanes having a Shore hardness of approximately 80 A-190 A and preferably about 85 A-110 A are suitable for use in extended inflow section 320. Other types of polymeric materials, as discussed above, can be used to form extended inflow section 320 but it is preferred that such materials provide an overall flexibility in the extended inflow section 320 higher than that of outflow section 310.

The physical dimensions (i.e. inner and outer diameters) of extended inflow section 320 are not the same as those discussed with respect to the inflow path 420 within the outflow section 310 in the preferred embodiments. It is desirable to utilize a comparatively larger diameter extended inflow path to minimize loss of flow due to friction over extended lengths, thus improving the flow capabilities of the catheter.

Polymeric tip plug 330 is shown at the distal most end of the catheter in FIG. 3. This soft insert, when present, is typically between about 0.25 and 5.0 mm in length and more preferably between about 1.5 and 2.0 mm. The diameter of the plug can be slightly smaller than the inner diameter of extended inflow section 320.

Plug 330 is preferable made of a softer polymer than that of distal section 520. Particularly suitable is the polyurethane copolymer sold as TECOFLEX having a Shore harness between about 70 A and 150 A, preferably between about 75 A and 95 A. Such a soft tip provides added mass to the blunt distal end of the catheter, promoting correct placement in the subarachnoid space with minimal trauma. This allows for shorter healing times of the entry wound and helps to minimize the risk of post-procedure infection.

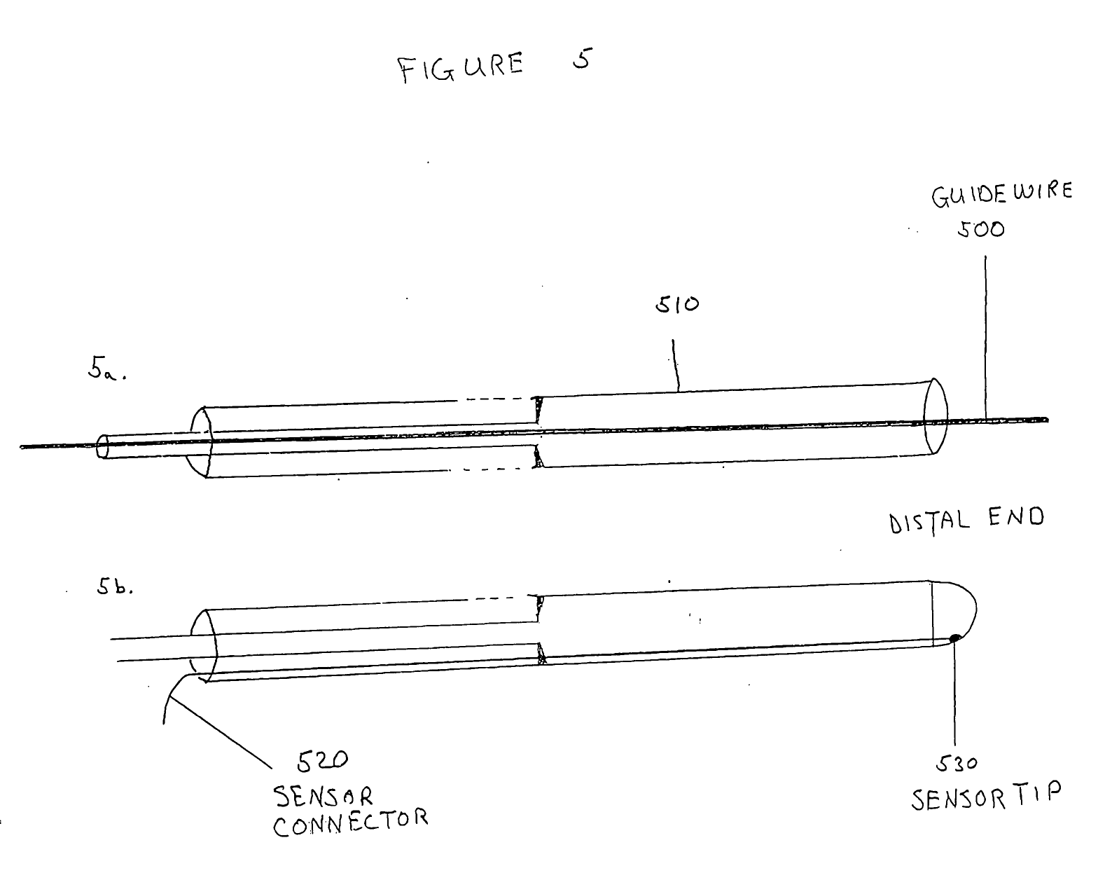

In other embodiments, the catheter can have an open end at its tip to allow placement of the catheter over a guide wire, FIG. 5a. A catheter guide wire 500 is first maneuvered to the desired location within the patient and then the open tipped catheter 510 is fed over the length of the guide wire. When the catheter has reached the correct location the guide wire 500 is removed.

Additionally, the catheters of the present invention may be made radio-opaque, if so desired, by the placement of rings or other markers of radio-opaque metals such as platinum, in or on the polymeric material at appropriate locations along the length of the catheter.

Additionally, the catheters of the present invention, FIG. 5b, can have one or more additional lumens, such as a lumen containing a sensor connector 520 and at sensor 530 to measure pressure or temperature within the subarachnoid space or containing a fiber optic sensor to visualize the site of injury and the subarachnoid space.

### Fenestrations

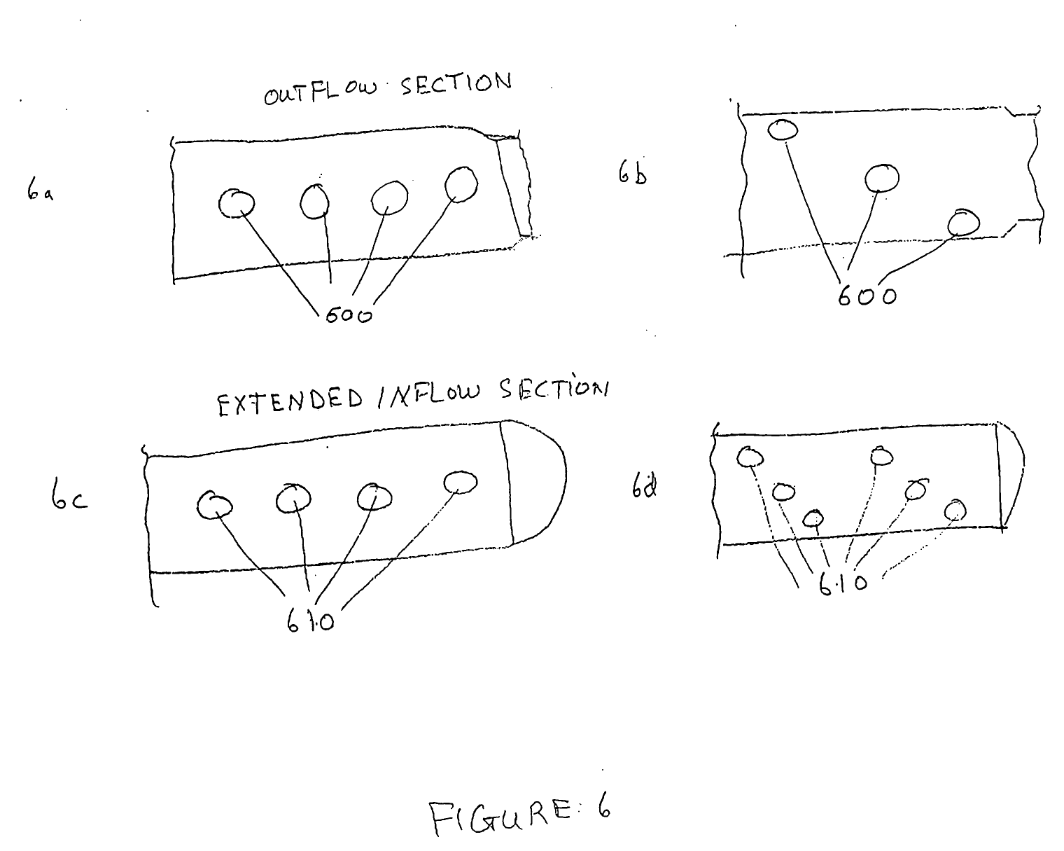

FIG. 6 illustrates the fenestrations 600 and 610 within the outflow 6a and 6b and extended inflow 6c and 6d sections of a catheter in accordance with the present invention. With respect to the size or diameter of outflow apertures 600, a relatively small diameter is preferred. In the preferred embodiments, apertures 600 typically have diameters in the range of about 0.25 mm to 1.5 mm depending upon the diameter of the catheter and the ability of the catheter to maintain the ACSF flow rates discussed above. With respect to the size or diameter of extended inflow apertures 610, a larger range of diameters is tolerated. Apertures 610 typically have diameters in the range of about 0.25 mm to 2.5 mm depending upon the diameter of the catheter and the ability of the catheter to maintain the ACSF flow rates discussed above. The extended inflow section can comprise a fabric containing micro-apertures. With respect to the aperture spacing, number, and pattern, it is within the scope of this invention that the apertures be arranged in a spiral or other nonlinear or random pattern, or variable spacing or diameters can be used.

Overall, aperture size, diameter, number, spacing, pattern, and shape are selected to obtain a useful or optimum flow rate for the transport of fluids such as ACSF, drugs, or diagnostics, to and from the subarachnoid space. Of course, the goal of optimizing fluid flow rate must also be balanced with maintaining structural integrity of the device.

Another consideration is that individual nerve root fibers of the cauda equina can partially or completely block fluid flow through one or more of apertures 600 and 610, hindering the efficiency of the catheter infusion and drainage function. The aperture characteristics are therefore chosen to reduce the likelihood of clogging from such neural structures. For example, nerve contact with the apertures can be prevented by broadening aperture suction zones and placing projections on the outer surface of the catheters in the region of the apertures. Such design features prevent or minimize clogging of apertures 600 and 610 when ACSF is transported at higher rates.

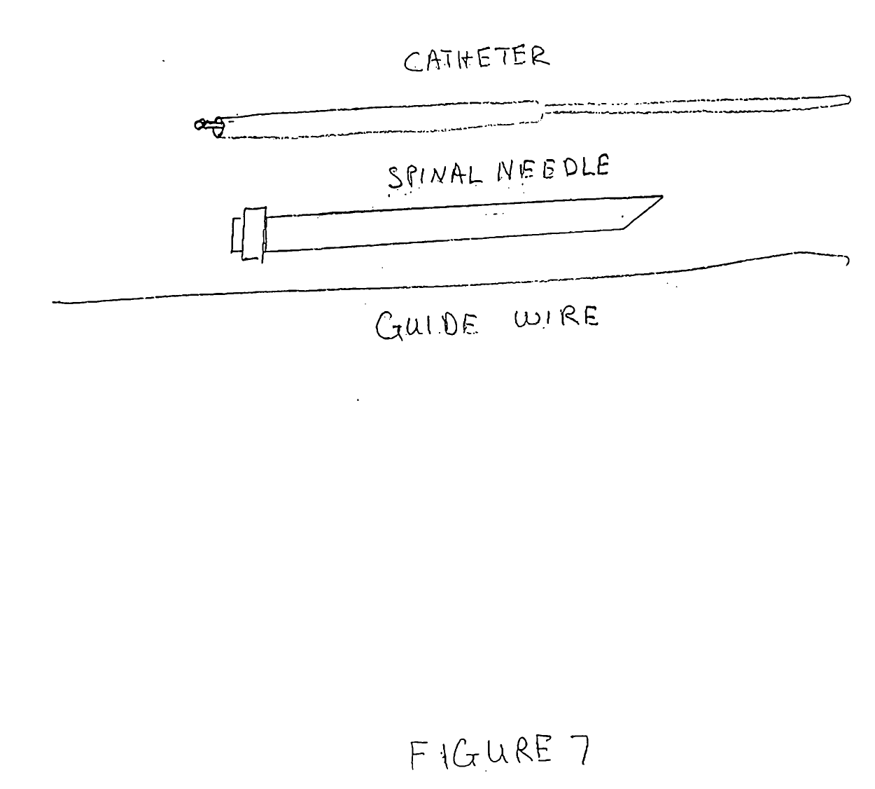

The catheter kit illustrated in FIG. 7 is preferably used to transport ACSF to and from the subarachnoid space. In an application, a large gauge spinal needle, for example, a Touhy needle, it typically first inserted into the subarachnoid space. An internal guide wire is insert into the catheter and then the catheter is inserted through the spinal needle and manipulated until its distal end is in the vicinity of the damaged neural tissue to be treated. The internal guide wire is then removed and flow established. Alternatively, a guide wire can be inserted through the spinal needle and manipulated to a target site. The catheter without a closed end tip can then be moved over the guide wire into position. Still other variations of the present invention include use of a catheter having a separate guide wire lumen.

The embodiments described above provide novel devices for fluidly communicating and circulating fluid with the subarachnoid space. It will be manifest that various materials, shapes, and modifications can be made without departing from the scope or spirit of the invention.

### EXAMPLES

A catheter in accordance with example 7 listed in the table below is designed to be compatible with a 14 gauge extra thin walled Touhy needle, limiting the outside diameters of the outflow and extended inflow sections to 1.68 mm. The length of the outflow section including the outflow fenestrations is 215 mm. The length of the extended inflow section including the inflow fenestrations is 120 mm useful for treatment of injury to the thoracic spine.

The outflow section is coaxial with the outflow lumen occupying the outside annular space and the inflow lumen occupying the central circular space. The outer wall section is 0.22 mm thick and contains a metal (Inox) coil that extends from the proximal end of the section to the outflow fenestrations. The inner partitioning wall is 0.13 mm thick and extends the entire length of the section. (The coiled outer wall provides the radial strength of resist kinking or crushing. The inner wall provides the longitudinal strength to resist tensiler forces and allows for a higher density of outflow fenestrations in the outflow section. The higher density of fenestrations reduces the outflow section length and associated fluid pressure loss.)

The extended inflow section contains a single inflow lumen. The section incorporates a conical connection that is the transition between the lumen diameter in the outflow section and the larger lumen diameter in the extended inflow section. The wall in the extended section is 0.17 mm thick and does not contain a coil.

The cross sectional area of the inflow lumen in 0.114 mm² in the outflow section and expands to 1.37 mm² in the extended section. The cross sectional area of the outflow lumen is 1.06 mm². The inflow and outflow lumen sizes have been balanced to provide a maximum flow rate of 15 mL/min within the 35 mmHg.

The outflow section contains 9 rows of 4 fenestrations each being 0.5 mm in diameter. The inflow section contains 6 rows of 4 fenestrations each being 0.75 mm in diameter.

Examples of the catheters of the present invention are provided in Table 1.

TABLE 1
Catheter Examples

| | | | | OUTFLOW | | | |
|---|---|---|---|---|---|---|---|
| | | | OUTFLOW | SECTION | | EXTENDED | |
| | OUTFLOW | OUTFLOW | SECTION | INFLOW | EXTENDED | INFLOW | |
| | SECTION | SECTION | LUMEN | LUMEN | INFLOW | LUMEN | MAXIMUM |
| EX. | LENGTH | DIAMETER | AREA | AREA | LENGTH | AREA | FLOW |
| NO. | (mm) | (mm) | (mm²) | (mm²) | (mm) | (mm²) | (mL/min) |
| 1 | 150 | 1.68 | 1.064 | 0.114 | 30 | 1.37 | 22 |
| 2 | 150 | 1.98 | 1.743 | 0.164 | 30 | 1.37 | 50 |
| 3 | 150 | 2.29 | 2.455 | 0.245 | 150 | 1.37 | 107 |
| 4 | 300 | 1.68 | 1.064 | 0.114 | 450 | 1.37 | 10 |
| 5 | 300 | 1.98 | 1.743 | 0.164 | 450 | 1.37 | 24 |
| 6 | 300 | 2.29 | 2.455 | 0.245 | 450 | 1.37 | 50 |
| 7 | 215 | 1.68 | 1.064 | 0.114 | 120 | 1.37 | 15 |
| 8 | 215 | 1.98 | 1.743 | 0.164 | 120 | 1.37 | 35 |
| 9 | 215 | 1.68 | 1.064 | 0.114 | 270 | 1.37 | 15 |
| 10 | 215 | 1.98 | 1.743 | 0.164 | 270 | 1.37 | 35 |
| 11 | 215 | 1.68 | 1.064 | 0.114 | 420 | 1.37 | 15 |
| 14 | 215 | 1.98 | 1.743 | 0.164 | 420 | 1.37 | 35 |
| 15 | 215 | 2.29 | 2.455 | 0.245 | 120 | 1.37 | 75 |

Definitions

The following terms shall have, for the purposes of this application, the respective meanings set forth below.

cerebrospinal tissue. Cerebrospinal tissue includes all tissues bathed by cerebrospinal fluid.

inserted portion. The inserted portion of a catheter is that portion of a catheter that is designed to be inserted into the subarachnoid space of a patient. In certain embodiments hereunder the inserted portion of a catheter can be the entire catheter.

minimum cross-sectional area. A cross-sectional area of a lumen is the area inside a cross-section of the lumen (that is perpendicular to the direction of flow of fluid within that lumen) through which fluid flowing through that lumen can flow. (Thus, a cross-sectional area of the outer of two concentric lumens does not include the area within the inner lumen.) The minimum cross-sectional area is the smallest cross-sectional area that exists for a particular lumen.

oncotic agent. By oncotic agent is meant substances, generally macromolecules, that are of a size that is not readily able to leave the body cavity or other fluid containing body spaces (such as the cerebrospinal pathway, including the cerebral ventricles and subarachnoid spaces) into which they are inserted. Such oncotic agents are exemplified by blood plasma expanders which are known in general as macromolecules having a size sufficient to inhibit their escape from the blood plasma through the circulatory capillary bed into the interstitial spaces of the body. Serum albumin, preferably human serum albumin, is one well known blood plasma protein that can be used as an oncotic agent. Polysaccharide blood plasma expanders are often glucan polymers. For example, Hetastarch (a product of American Home Products) is an artificial colloid derived from a waxy starch composed almost entirely of amylopectin with hydroxyethyl ether groups introduced into the alpha (1-4) linked glucose units. The colloid properties of a 6% solution (wt/wt) of hetastarch approximate that of human serum albumin. Other polysaccharide derivatives can be suitable as oncotic agents in the blood substitute according to the invention. Among such other polysaccharide derivatives are hydroxymethyl alpha (1-4) or (1-6) polymers and cyclodextrins. In general, it is preferred that the polysaccharide is one that is non-antigenic. High molecular weight agents such as Dextran 70 having a molecular weight of about 70,000 Daltons are generally less preferred because they increase viscosity of the colloidal solution and impair the achievement of high flow rates. Preferably, the oncotic agent is in an amount effective to provide, in conjunction with other components of a fluorocarbon nutrient emulsion or a nutrient solution, an oncotic pressure of one to seven torr.

Where noted above, publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in its entirety in the manner described above for publications and references.

While this invention has been described with an emphasis upon preferred embodiments, it will be obvious to those of ordinary skill in the art that variations in the preferred devices and methods can be used and that it is intended that the invention can be practiced otherwise than as specifically described herein. Accordingly, this invention includes all modifications encompassed within the spirit and scope of the invention as defined by the claims that follow.

That which is claimed is:

1. A multi-lumen intrathecal catheter capable of use for circulating a fluid through a portion of the spinal cord of a patient and having an insertable portion adapted for insertion into the spinal cord, comprising:
a first lumen having a distal and a proximal end, the proximal end being adapted to being connected to a reservoir containing a fluid;
a first fluid exchange section connected to the distal end of the first lumen having at least one inlet;
a second lumen having a distal and a proximal end; and
a second fluid exchange section connected to the distal end of the second lumen having at least one outlet,
wherein the at least one inlet and the at least one outlet are offset from each other.

2. The catheter of claim 1, wherein the offset is at least three centimeters.

3. The catheter of claim 1, wherein said first lumen has a length of 335 mm;
wherein said second lumen has a length of 215 mm; and
wherein the tip of the distal end of the first lumen and the tip of the distal end of the second lumen are offset from each other by 120 mm.

4. The catheter of claim 1, wherein the minimum cross-sectional area of the first lumen is smaller than the minimum cross-sectional area of the second lumen.

5. The catheter of claim 1, further comprising a third lumen for measuring pressure.

6. The catheter of claim 5 wherein the third lumen is a fiber optic lumen.

7. The catheter of claim 1, wherein the tip of at least one lumen is non-traumatic.

8. The catheter of claim 1, wherein the tip of at least one lumen is an open tip.

9. The catheter of claim 1, wherein the tip of at least one lumen is closed and the at least one inlet and the at least one outlet comprise a plurality of fenestrations.

10. The catheter of claim 9 wherein each fenestration is no more than 2.5 millimeters in diameter.

11. The catheter of claim 9, wherein each fenestration is no more than 1.25 millimeters in diameter

12. The catheter of claim 9, wherein each fenestration is between 0.25 and 2.5 millimeters in diameter.

13. The catheter of claim 1, wherein the diameter of the inserted portion of the catheter does not exceed two millimeters.

14. The catheter of claim 1, wherein the diameter of the inserted portion of the catheter does not exceed three millimeters.

15. The catheter of claim 1, wherein the diameter of the inserted portion of the catheter does not exceed a diameter equal to a twelve gauge needle.

16. The catheter of claim 1, wherein the diameter of the inserted portion of the catheter does not exceed a diameter equal to a fourteen gauge needle.

17. The catheter of claim 1, wherein the diameter of the inserted portion of the catheter is at least two millimeters.

18. The catheter of claim 1, wherein the diameter of the inserted portion of the catheter is between two and four millimeters.

19. A kit containing a catheter in accordance with claim 1, comprising a catheter, a spinal needle, and a guide wire.

20. A method of irrigating a portion of the subarachnoid space of a patient utilizing a catheter in accordance with claim 1, comprising:
(a) inserting the catheter into the subarachnoid space of the patient;
(b) infusing a fluid into the subarachnoid space through a first lumen of the catheter; and
(c) removing the fluid through a second lumen of the catheter.

21. The method of claim 20 wherein said irrigation of the subarachnoid space is effective to remove blood products.

22. The method of claim 20 wherein said irrigation of the subarachnoid space is effective to cool tissue contacting the subarachnoid space.

23. The method of claim 20 wherein said irrigation of the subarachnoid space is effective to deliver a therapeutic agent.
